(19) United States
(12) Patent Application Publication
(10) Pub. No.: US 2023/0030989 A1
(43) Pub. Date: Feb. 2, 2023

(54) JUST SUNSCREEN

(71) Applicant: Pouch Pac Innovations, LLC, Sarasota, FL (US)

(72) Inventors: R. Charles Murray, Sarasota, FL (US); John Harlin, Sarasota, FL (US)

(21) Appl. No.: 17/636,668

(22) PCT Filed: Aug. 21, 2020
(86) PCT No.: PCT/US2020/047403

§ 371 Date: Feb. 18, 2022

Related U.S. Application Data
(60) Provisional application No. 62/889,733, filed on Aug. 21, 2019.

(51) Int. Cl. A61K 8/27; A61K 8/29; A61K 8/92; A61Q 17/04; A61K 8/55; A61K 8/26; A61K 8/37

(52) U.S. Cl. CPC A61K 8/27; A61K 8/29; A61K 8/927; A61K 8/92; A61Q 17/04; A61K 8/553; A61K 8/26; A61K 8/375; A61K 2800/262

(57) ABSTRACT

A topical sunscreen composition including a lotion having titanium dioxide, zinc oxide, water, avocado oil, sunflower oil, bee's wax, coconut oil, monolaurin, stearyl dimethicone, cetylesters, ethyl linoleate, triethyl citrate, fragrance and Lecithin. In another aspect, there is disclosed a topical sunscreen composition including a lotion having zinc oxide, water, avocado oil, zeolite, bee's wax, coconut oil, monolaurin, ethyl linoleate, bentonite, tri ethyl citrate, fragrance and Lecithin.

### CROSS REFERENCE TO RELATED APPLICATION

This application claims priority to U.S. Application Ser. No. 62/889,733 filed on Aug. 21, 2019, the entire disclosure of which is hereby incorporated by reference.

### FIELD OF THE INVENTION

The instant application is directed to a formulation for a topical product with sunscreen.

### BACKGROUND OF THE INVENTION

Sunscreens are recommended to be applied when a person is exposed to the sun to prevent potential skin damage. Some sunscreens include ingredients that are harmful to the environment and less than ideal to apply to a person. There is therefore a need in the art for a natural sunscreen product.

### SUMMARY OF THE INVENTION

In one aspect, there is disclosed a topical sunscreen composition including a lotion having titanium dioxide, zinc oxide, water, avocado oil, sunflower oil, bee's wax, coconut oil, monolaurin, stearyl dimethicone, cetyl esters, ethyl linoleate, triethyl citrate, fragrance and Lecithin.

In another aspect, there is disclosed a topical sunscreen composition including a lotion having zinc oxide, water, avocado oil, zeolite, bee's wax, coconut oil, monolaurin, ethyl linoleate, bentonite, triethyl citrate, fragrance and Lecithin.

Also disclosed is a method of protecting a body from sun exposure which includes applying the lotion compositions to a body and exposing insects to the lotion. Additionally, the lotion composition includes an SPF factor of greater than 30 and is a broad spectrum sunscreen.

Additional features and advantages of mosquito control products described herein will be set forth in the detailed description which follows, and in part will be readily apparent to those skilled in the art from that description or recognized by practicing the embodiments described herein, including the detailed description which follows, the claims, as well as the appended drawings.

It is to be understood that both the foregoing general description and the following detailed description describe various embodiments and are intended to provide an overview or framework for understanding the nature and character of the claimed subject matter. The accompanying drawings are included to provide a further understanding of the various embodiments, and are incorporated into and constitute a part of this specification. The drawings illustrate the various embodiments described herein and together with the description serve to explain the principles and operations of the claimed subject matter.

### BRIEF DESCRIPTION OF THE DRAWINGS

FIG. 1 is a graph of the absorbance as a function of the wavelength for a critical wavelength determination.

### DETAILED DESCRIPTION OF THE EMBODIMENTS

In one aspect, there is disclosed herein a formulation that may be utilized as a topical lotion and provide sunscreen protection. The formulation may include organic and chemical-free compositions that will provide sunscreen protection.

In one aspect the composition may include titanium dioxide, zinc oxide, water, avocado oil, Zeolite, bentonite, sunflower oil, bee's wax, coconut oil, monolaurin, stearyl dimethicone, cetyl esters, ethyl linoleate, triethyl citrate, fragrance and Lecithin. Various amounts of these materials may be present in the composition as will be described in more detail below.

As described above, the composition may include active ingredients and inactive ingredients. The titanium dioxide and zinc oxide may be classified as active ingredients wherein the water, avocado oil, zeolite, bentonite, sunflower oil, bee's wax, coconut oil, monolaurin, stearyl dimethicone, cetyl esters, ethyl linoleate, triethyl citrate, fragrance and Lecithin may be considered inert or inactive ingredients.

### Example 1

In one aspect, the lotion formulation may include a sunscreen with a SPF value of 30. The formulation may include:

| | | | DENSITY | |
|---|---|---|---|---|
| | DENSITY | Wt % | X % | PERCENT RANGE |
| Active Ingredient | | | | |
| Titanium Dioxide | 3.9 | 6 | 0.234 | PLUS-MINUS 33% |
| Zinc Oxide | 5.61 | 6 | 0.3366 | PLUS-MINUS 45% |
| Inactive Ingredients | | | | |
| Water | 1 | 33 | 0.33 | PLUS-MINUS 35% |
| Avocado Oil | 0.92 | 28 | 0.2576 | PLUS-MINUS 33% |
| Sunflower Oil | 0.89 | 7.3 | 0.06497 | PLUS-MINUS 40% |
| Beeswax | 0.961 | 4.3 | 0.041323 | PLUS-MINUS 30% |
| coconut oil | 0.935 | 3 | 0.02805 | PLUS-MINUS 33% |
| Monolaurin | 0.925 | 3 | 0.02775 | PLUS-MINUS 33% |
| Stearyl Dimethicone | 0.82 | 3 | 0.0246 | PLUS-MINUS 33% |
| Cetyl Esters | 0.873 | 2 | 0.01746 | PLUS-MINUS 33% |
| Ethyl Linoleate | 0.876 | 2 | 0.01752 | PLUS-MINUS 33% |
| TriEthyl Citrate | 1.14 | 2 | 0.0228 | PLUS-MINUS 15% |
| Fragrance | 0.885 | 0.2 | 0.00177 | PLUS-MINUS 50% |
| Lecithin | 1.52 | 0.2 | 0.00304 | PLUS-MINUS 25% |

As specified above, the composition may include titanium dioxide, zinc oxide, water, avocado oil, sunflower oil, bee's wax, coconut oil, monolaurin, stearyl dimethicone, cetyl esters, ethyl linoleate, triethyl citrate, fragrance and Lecithin.

The titanium dioxide may be present in an amount of from 4 to 8% by weight. The zinc oxide may be present in an amount of from 3 to 9% by weight. The water may be present in an amount of from 21 to 44% by weight. The avocado oil may be present from 19 to 37% by weight. The sun flower oil may be present in an amount of from 4% to 10% by weight. The bee's wax may be present in an amount of from 3% to 6% by weight. The coconut oil may also be present in an amount of from 2 to 4% by weight. The monolaurin may be present in an amount of from 2 to 4% by weight. The steryl dimethicone may be present in an amount of from 2 to 4% by weight. The cetyl esters may be present in an amount of from 1 to 3% by weight. The ethyl linoleate may be present in an amount of from 1 to 3% by weight. The triethyl citrate may be present in an amount of 1.5 to 2.5% by weight. The fragrance may be present in an amount of 1 to 3% by weight. Additionally, the composition may include powdered ingredients such as Lecithin present at about 0.5% by weight. The weight percentages are based on a total weight of the composition.

The composition of example 1 was made according to the following procedure. The avocado oil and sunflower oils were heated from room temperature to 145 to 160 degrees F. in a mixing vat under agitation. The lecithin and water were added to the composition when the temperature approaches 100 F. The coconut oil, monolaurin, cetyl esters, triethyl citrate, ethyl linoleate and bee's wax are next added. The mixture is heated and agitated until about 145 degrees F. and a shearing mechanism is activated and maintained until the mixture contains no grit. Next, stearyl dimethicone, zinc oxide and titanium oxide are added slowly and again the shearing mechanism is activated and maintained until the mixture contains no grit. The composition is maintained under agitation and heat is tuned off from the composition. Fragrance is added and the composition cools to about 125-130 F. The composition is agitated and sheared for a specified time dependent upon the size of the batch. After which time the lotion may be packaged.

Sunscreen Testing

The sunscreen formulation as presented above was tested for efficacy following an FDA protocol. Data from the testing is presented below in Table 2.

TABLE 2

| | | | | | | | STD | |
|---|---|---|---|---|---|---|---|---|
| Subject | | MED/ | I | Skin | MED I | MED II | (7% PadO/ | SPF |
| ID # | Sex | Hr | (Amps) | Type | J/m² | J/m² | 3% Oxyb) | Value |
| 46 0596 | F | 128.5 | 6.0 | II | 137.50 | 137.50 | 16.30 | 30.00 |
| 44 6325 | F | 126.7 | 6.0 | II | 110.00 | 110.00 | 16.30 | 34.50 |
| 74 4514 | F | 127.5 | 6.0 | II | 137.50 | 137.50 | 16.30 | 34.50 |
| 48 5910 | M | 129.8 | 6.0 | II | 110.00 | 110.00 | 18.75 | 34.50 |
| 50 5281 | F | 80.0 | 6.0 | I | 176.00 | 176.00 | 16.30 | 34.50 |
| 74 4514 | F | 127.2 | 6.0 | I | 137.50 | 137.50 | 16.30* | 39.60* |
| 60 7230 | F | 80.0 | 6.0 | II | 171.90 | 171.90 | 16.30 | 39.67 |
| 38 9648 | M | 125.9 | 6.0 | II | 137.50 | 137.50 | 18.75 | 34.50 |
| 56 1787 | F | 126.9 | 6.0 | II | 110.00 | 110.00 | 16.30 | 34.50 |
| 58 5653 | F | 128.2 | 6.0 | II | 110.00 | 110.00 | 16.30 | 34.50 |
| 48 4004 | F | 80.0 | 6.0 | II | 137.50 | 137.50 | 16.30 | 34.50 |

| | | |
|---|---|---|
| MEAN (x) | 16.79 | 34.57 |
| STANDARD DEV (s) | 1.03 | 2.28 |
| STD. ERROR | 0.33 | 0.72 |
| S.E. % OF MEAN | 1.97 | 2.08 |
| N | 10 | 10 |
| UPPER 5% t DIST. | 2.2622 | 1.8331 |
| A VALUES | 0.7465 | 1.3198 |
| LABEL SPF | 16 | 33 |

MED: Minimal Erythemal Dose
I: Intensity of light source

The Sun Protection Factor (SPF) of the test material when tested on ten subjects under static conditions yielded the mean SPF value of 34.57 and the label SPF of 33. The mean SPF of the 7% Padimate 0/3% Oxybenzone standard on the same panel was 16.79 and was within the standard deviation range of the expected SPF of 16.3+/−3.43.

A patch test was also performed for the sunscreen formulation presented above.

Subjects reported to the testing facility, at which time inclusion/exclusion criteria was reviewed and Informed Consent obtained. Upon verification of eligibility to enroll, the test articles were applied to the upper arm of each subject. The test articles were applied to the Webril□ disks according to a dose regime (see Section 5.1). The skin was marked with dots of crystal violet applied on either side of the top disk and below the bottom disk of the strip to enable exact location of subsequent patches. Each subject was asked to avoid the dye marks during washing and to keep the patches dry.

Subjects received patches containing the test article and two standard controls which were applied as 0.2 ml/0.2 g samples in one of five randomly assigned orders on the upper arm. The sample order for each subject was documented and maintained throughout the study in the form of a color-coded identification card. Subjects were instructed to keep the patches dry and in place for 23 hours then to remove and discard them and to return to the test center one hour later. Patches were applied on Days 1, 2, 3 and 4. Patches were applied to the same site on each day unless a reaction stronger than a mild erythema (i.e. greater than a score of 1.5 (Appendix 5)) was present in which case the patch strip was cut and the relevant patch (es) not reapplied. Assessment of patch sites was immediately before application of the next patch on Days 2, 3, 4 and on Day 5. After removal of the patches the test sites were wiped with a damp tissue to remove any remaining product.

The data from the testing is presented in Table 3 below.

Data from Test Formulation

| Subject No | Code | Day 2 | Day 3 | Day 4 | Day 5 | comments |
|---|---|---|---|---|---|---|
| 1 | 1 | 0.0 | 0.0 | 0.0 | 0.0 | |
| 2 | 1 | 0.0 | 0.0 | 0.0 | 0.0 | |
| 3 | 1 | 0.0 | 0.0 | 0.0 | 0.0 | |
| 4 | 1 | 0.0 | 0.0 | 0.0 | 0.0 | A on day 3, |
| | | | | | | 4 and 5 |
| 5 | 1 | 0.0 | 0.0 | 0.0 | 0.0 | |
| 6 | 1 | 0.0 | 0.0 | 0.0 | 0.0 | |
| 7 | 1 | 0.0 | 0.0 | 0.0 | 0.0 | A on day 4 |
| | | | | | | and 5 |
| 8 | 1 | 0.0 | 0.0 | 0.0 | 0.0 | |
| 9 | 1 | 0.0 | 0.0 | 0.0 | 0.0 | |
| 10 | 1 | 0.0 | 0.0 | 0.0 | 0.0 | A on day 4 |
| | | | | | | and 5 |
| 11 | 1 | 0.0 | 0.0 | 0.0 | 0.0 | |
| 12 | 1 | 0.0 | 0.0 | 0.0 | 0.0 | |
| 13 | 1 | 0.0 | 0.0 | 0.0 | 0.0 | A on day 5 |
| 14 | 1 | 0.0 | 0.0 | 0.0 | 0.0 | |
| 15 | 1 | 0.0 | 0.0 | 0.0 | 0.0 | |
| 16 | 1 | 0.0 | 0.0 | 0.0 | 0.0 | |
| 17 | 1 | 0.0 | 0.0 | 0.0 | 0.0 | A on day 4 |
| | | | | | | and 5 |
| 18 | 1 | 0.0 | 0.0 | 0.0 | 0.0 | |
| 19 | 1 | 0.0 | 0.0 | 0.0 | 0.0 | |
| 20 | 1 | 0.0 | 0.0 | 0.0 | 0.0 | A on day 4 |
| | | | | | | and 5 |
| 21 | 1 | 0.0 | 0.0 | 0.0 | 0.0 | |
| 22 | 1 | 0.0 | 0.0 | 0.0 | 0.0 | A on day 4 |
| | | | | | | and 5 |
| 23 | 1 | 0.0 | 0.0 | 0.0 | 0.0 | |
| 24 | 1 | 0.0 | 0.0 | 0.0 | 0.0 | |
| 25 | 1 | 0.0 | 0.0 | 0.0 | 0.0 | |
| 26 | 1 | 0.0 | 0.0 | 0.0 | 0.0 | A on day 5 |
| 27 | 1 | 0.0 | 0.0 | 0.0 | 0.0 | |
| 28 | 1 | 0.0 | 0.0 | 0.0 | 0.0 | |
| 29 | 1 | 0.0 | 0.0 | 0.0 | 0.0 | |
| 30 | 1 | 0.0 | 0.0 | 0.0 | 0.0 | A on day 3, |
| | | | | | | 4 and 5 |
| MEAN | | 0.0 | 0.0 | 0.0 | 0.0 | |
| STDEV | | 0.0 | 0.0 | 0.0 | 0.0 | |

### APPENDIX 7: INDIVIDUAL RESPONSES TO TEST ARTICLE 2—NEGATIVE CONTROL (DEIONISED WATER)

| Subject No | Code | Day 2 | Day 3 | Day 4 | Day 5 | comments |
|---|---|---|---|---|---|---|
| 1 | 2 | 0.0 | 0.0 | 0.0 | 0.0 | |
| 2 | 2 | 0.0 | 0.0 | 0.0 | 0.0 | |
| 3 | 2 | 0.0 | 0.0 | 0.0 | 0.0 | |
| 4 | 2 | 0.0 | 0.0 | 0.0 | 0.0 | A on day 3, |
| | | | | | | 4 and 5 |
| 5 | 2 | 0.0 | 0.0 | 0.0 | 0.0 | |
| 6 | 2 | 0.0 | 0.0 | 0.0 | 0.0 | |
| 7 | 2 | 0.0 | 0.0 | 0.0 | 0.0 | A on day 4 |
| | | | | | | and 5 |
| 8 | 2 | 0.0 | 0.0 | 0.0 | 0.0 | |
| 9 | 2 | 0.0 | 0.0 | 0.0 | 0.0 | |
| 10 | 2 | 0.0 | 0.0 | 0.0 | 0.0 | A on day 4 |
| | | | | | | and 5 |
| 11 | 2 | 0.0 | 0.0 | 0.0 | 0.0 | |
| 12 | 2 | 0.0 | 0.0 | 0.0 | 0.0 | |
| 13 | 2 | 0.0 | 0.0 | 0.0 | 0.0 | A on day 5 |
| 14 | 2 | 0.0 | 0.0 | 0.0 | 0.0 | |
| 15 | 2 | 0.0 | 0.0 | 0.0 | 0.0 | |
| 16 | 2 | 0.0 | 0.0 | 0.0 | 0.0 | |
| 17 | 2 | 0.0 | 0.0 | 0.0 | 0.0 | A on day 4 |
| | | | | | | and 5 |
| 18 | 2 | 0.0 | 0.0 | 0.0 | 0.0 | |
| 19 | 2 | 0.0 | 0.0 | 0.0 | 0.0 | |
| 20 | 2 | 0.0 | 0.0 | 0.0 | 0.0 | A on day 4 |
| | | | | | | and 5 |
| 21 | 2 | 0.0 | 0.0 | 0.0 | 0.0 | |
| 22 | 2 | 0.0 | 0.0 | 0.0 | 0.0 | A on day 4 |
| | | | | | | and 5 |
| 23 | 2 | 0.0 | 0.0 | 0.0 | 0.0 | |
| 24 | 2 | 0.0 | 0.0 | 0.0 | 0.0 | |
| 25 | 2 | 0.0 | 0.0 | 0.0 | 0.0 | |
| 26 | 2 | 0.0 | 0.0 | 0.0 | 0.0 | A on day 5 |
| 27 | 2 | 0.0 | 0.0 | 0.0 | 0.0 | |
| 28 | 2 | 0.0 | 0.0 | 0.0 | 0.0 | |
| 29 | 2 | 0.0 | 0.0 | 0.0 | 0.0 | |
| 30 | 2 | 0.0 | 0.0 | 0.0 | 0.0 | A on day 3, |
| | | | | | | 4 and 5 |
| MEAN | | 0.0 | 0.0 | 0.0 | 0.0 | |
| STDEV | | 0.0 | 0.0 | 0.0 | 0.0 | |

### APPENDIX 8: INDIVIDUAL RESPONSES TO TEST ARTICLE 3—POSITIVE CONTROL (0.3% W/V SLS)

| Subject No | Code | Day 2 | Day 3 | Day 4 | Day 5 | comments |
|---|---|---|---|---|---|---|
| 1 | 3 | 0.0 | 0.5 | 0.5 | 1.0 | |
| 2 | 3 | 0.0 | 0.0 | 0.0 | 0.0 | |
| 3 | 3 | 0.5 | 0.5 | 1.0 | 1.0 | |
| 4 | 3 | 0.0 | 1.0 | 1.0 | 1.5 | A on day 3, |
| | | | | | | 4 and 5, |
| | | | | | | P on day 5 |
| 5 | 3 | 0.0 | 0.0 | 0.0 | 0.0 | |
| 6 | 3 | 0.0 | 0.0 | 0.0 | 0.0 | |
| 7 | 3 | 0.5 | 1.0 | 2.0 | 2.0 | X on day 5, |
| | | | | | | A on day 4 |
| | | | | | | and 5 |
| 8 | 3 | 0.0 | 0.0 | 0.0 | 0.0 | |
| 9 | 3 | 0.0 | 0.5 | 1.0 | 1.0 | |
| 10 | 3 | 0.5 | 1.5 | 2.0 | 2.0 | X on day 5, |
| | | | | | | A on day 4 |
| | | | | | | and 5 |
| 11 | 3 | 0.0 | 0.5 | 1.0 | 1.5 | |
| 12 | 3 | 0.0 | 0.0 | 0.0 | 0.0 | |
| 13 | 3 | 0.0 | 0.5 | 1.0 | 1.0 | A on day 5 |
| 14 | 3 | 0.0 | 0.0 | 0.0 | 0.0 | |
| 15 | 3 | 0.0 | 0.5 | 0.5 | 1.0 | |
| 16 | 3 | 0.0 | 0.0 | 0.0 | 0.0 | |
| 17 | 3 | 0.5 | 1.0 | 1.5 | 2.0 | A on day 4 |
| | | | | | | and 5 |
| 18 | 3 | 0.0 | 0.0 | 0.5 | 1.0 | |
| 19 | 3 | 0.0 | 0.0 | 0.0 | 0.0 | |
| 20 | 3 | 0.0 | 0.5 | 1.0 | 1.5 | A on day 4 |
| | | | | | | and 5 |
| 21 | 3 | 0.0 | 0.0 | 0.5 | 1.0 | |
| 22 | 3 | 0.5 | 1.0 | 2.0 | 1.5 | X on day 5, |
| | | | | | | A on day 4 |
| | | | | | | and 5 |
| 23 | 3 | 0.0 | 0.0 | 0.0 | 0.0 | |
| 24 | 3 | 0.0 | 0.5 | 1.0 | 1.0 | |
| 25 | 3 | 0.0 | 0.0 | 0.0 | 0.0 | |
| 26 | 3 | 0.0 | 0.5 | 1.0 | 2.0 | A on day 5 |
| 27 | 3 | 0.0 | 0.0 | 0.0 | 0.0 | |
| 28 | 3 | 0.0 | 0.0 | 0.0 | 0.0 | |
| 29 | 3 | 0.0 | 0.5 | 0.5 | 1.0 | |
| 30 | 3 | 0.0 | 0.5 | 1.0 | 1.5 | A on day 3, |
| | | | | | | 4 and 5 |
| MEAN | | 0.08 | 0.37 | 0.63 | 0.82 | |
| STDEV | | 0.19 | 0.41 | 0.66 | 0.75 | |

The test samples can be considered [symbol] Safe for Use with claims as [symbol] Dermatologically tested,∞ ∞Clinically Proven,∞ ∞Kind to Skin,∞ ∞Mild for skin,∞ and ∞Safe for Skin∞ are all substantiated.

Additionally, the broad spectrum properties of the formula as presented above was evaluated.

The critical wavelength was found to be 387.59 nm, and satisfies the required minimum of 370 nm for Broadscreen Spectrum labeling.

### Example 2

In one aspect, the lotion formulation may include a sunscreen with a SPF value of 30. The formulation may include:

| | DENSITY | Wt % | PERCENT RANGE |
|---|---|---|---|
| Active Ingredient | | | |
| Zinc Oxide | 5.61 | 7 | PLUS-MINUS 45% |
| Inert Ingredients | | | |
| Water | 1 | 30.4 | PLUS-MINUS 35% |
| Avocado Oil | 0.92 | 29 | PLUS-MINUS 33% |
| Zeolite | 2.2 | 12 | PLUS-MINUS 40% |
| Beeswax | 0.961 | 3 | PLUS-MINUS 30% |
| coconut oil | 0.935 | 12 | PLUS-MINUS 33% |
| Monolaurin | 0.925 | 12 | PLUS-MINUS 33% |
| bentonite | 2.5 | 6 | PLUS-MINUS 33% |
| Ethyl Linoleate | 0.876 | 2 | PLUS-MINUS 33% |
| TriEthyl Citrate | 1.14 | 2 | PLUS-MINUS 15% |
| Fragrance | 0.885 | 0.3 | PLUS-MINUS 50% |
| Lecithin | 1.52 | 0.3 | PLUS-MINUS 25% |

As specified above, the composition may include zinc oxide, water, avocado oil, zeolite, bee [symbol] s wax, coconut oil, monolaurin, ethyl linoleate, bentonite, triethyl citrate, fragrance and Lecithin.

The zinc oxide may be present in an amount of from 3.85 to 10.15% by weight. The water may be present in an amount of from 20 to 41% by weight. The avocado oil may be present from 19 to 39% by weight. The zeolite may be present in an amount of from 7% to 17% by weight. The bee [symbol] s wax may be present in an amount of from 2% to 4% by weight. The coconut oil may also be present in an amount of from 8 to 16% by weight. The monolaurin may be present in an amount of from 8 to 16% by weight. The bentonite may be present in an amount of from 4% to 8% by weight. The ethyl linoleate may be present in an amount of from 1 to 3% by weight. The triethyl citrate may be present in an amount of 1.5 to 2.5% by weight. The fragrance may be present in an amount of 0.1 to 0.5% by weight. Additionally, the composition may include powdered ingredients such as Lecithin present at about 0.2 to 0.4% by weight. The weight percentages are based on a total weight of the composition. The sun screen composition of example 2 is clear or transparent when applied to a person.

The composition of example 2 was made according to the following procedure. The avocado oil was heated from room temperature to 145 to 160 degrees F. in a mixing vat under agitation. The lecithin and water were added to the composition when the temperature approaches 100 F. The coconut oil, monolaurin, cetyl esters, triethyl citrate, ethyl linoleate and bee [symbol] s wax are next added. The mixture is heated and agitated until about 145 degrees F. and a shearing mechanism is activated and maintained until the mixture contains no grit. Next, zinc oxide, bentonite and zeolite are added slowly and again the shearing mechanism is activated and maintained until the mixture contains no grit. The composition is maintained under agitation and heat is tuned off from the composition. Fragrance is added and the composition cools to about 125-130 F. The composition is agitated and sheared for a specified time dependent upon the size of the batch. After which time the lotion may be packaged.

Sunscreen Testing

The sunscreen formulation as presented above was tested for efficacy following an FDA protocol. Data from the testing is presented below in Table 4.

The broad spectrum properties of the formula as presented above was evaluated.

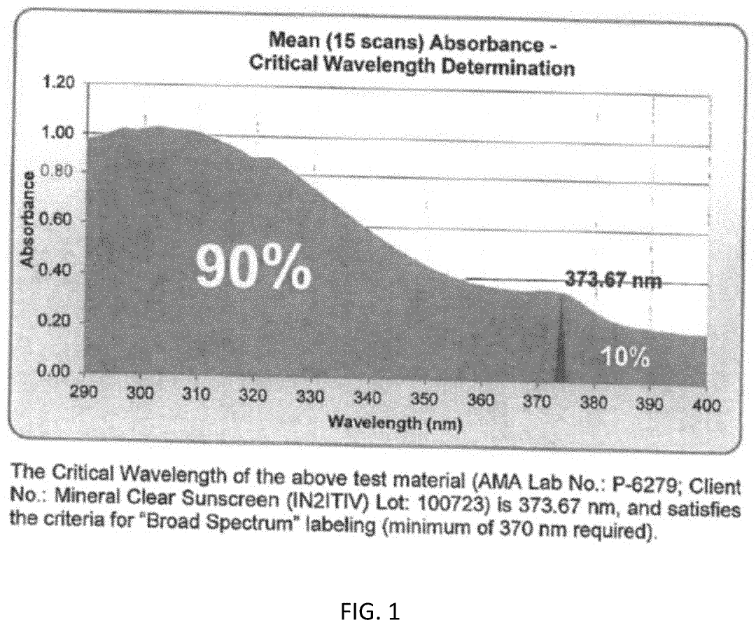

The critical wavelength was found to be 373.67 nm, and satisfies the required minimum of 370 nm for Broadscreen Spectrum labeling, as shown in Table 4 and as denoted in FIG. 1.

TABLE 4
critical wave length: (requirement: minimum λc = 370 nm
Critical Wavelength values After Pre-irradiation Procedure 7.0 MED/h
UV Source (LS1000-6S-UV Solar Simulator) Irradiance
Irradiation Time (Single Plate): 2057 sec

| | Location | Location | Location | Location | Location |
|---|---|---|---|---|---|
| Plate 1 | 373 | 374 | 373 | 374 | 373 |
| Plate 2 | 373 | 374 | 374 | 374 | 374 |
| Plate 3 | 374 | 374 | 374 | 374 | 373 |

| | |
|---|---|
| Average | 373.67 |

We claim:

1. A topical sunscreen composition comprising:
a lotion including titanium dioxide, zinc oxide, water, avocado oil, sunflower oil, bee's wax, coconut oil, monolaurin, stearyl dimethicone, cetyl esters, ethyl linoleate, triethyl citrate, fragrance and Lecithin.

2. The topical sunscreen composition of claim 1 wherein the titanium dioxide is present in an amount of from 4 to 8% by weight, the zinc oxide is present in an amount of from 3 to 9% by weight, the water is present in an amount of from 21 to 44% by weight, the avocado oil is present in an amount of from 19 to 37% by weight, the sun flower oil is present in an amount of from 4% to 10% by weight, the bee's wax is present in an amount of from 3% to 6% by weight, the coconut oil is present in an amount of from 2 to 4% by weight, the monolaurin is present in an amount of from 2 to 4% by weight, the steryl dimethicone is present in an amount of from 2 to 4% by weight, the cetyl esters is present in an amount of from 1 to 3% by weight, the ethyl linoleate is present in an amount of from 1 to 3% by weight, the triethyl citrate is present in an amount of 1.5 to 2.5% by weight, the fragrance is present in an amount of 1 to 3% by weight, the Lecithin is present at about 0.5% by weight wherein the weight is based upon a total weight of the composition.

3. The topical sunscreen composition of claim 1 wherein the topical sunscreen composition has an SPF of 30 or greater.

4. The topical sunscreen composition of claim 1 wherein the topical sunscreen composition is a broad spectrum sunscreen.

5. A topical sunscreen composition comprising: zinc oxide, water, avocado oil, zeolite, bee's wax, coconut oil, monolaurin, ethyl linoleate, bentonite, triethyl citrate, fragrance and Lecithin.

6. The topical sunscreen composition of claim 5 wherein the zinc oxide is present in an amount of from 3.85 to 10.15% by weight, the water is present in an amount of from 20 to 41% by weight, the avocado oil is present from 19 to 39% by weight, the zeolite is present in an amount of from 7% to 17% by weight, the bee's wax is present in an amount of from 2% to 4% by weight, the coconut oil is present in an amount of from 8 to 16% by weight, the monolaurin is present in an amount of from 8 to 16% by weight, the bentonite is present in an amount of from 4% to 8% by weight, the ethyl linoleate is present in an amount of from 1 to 3% by weight, the triethyl citrate is present in an amount of 1.5 to 2.5% by weight, the fragrance is present in an amount of 0.1 to 0.5% by weight, the Lecithin is present at about 0.2 to 0.4% by weight wherein the weight is based upon a total weight of the composition.

7. The topical sunscreen composition of claim 5 wherein the topical sunscreen composition has an SPF of 30 or greater.

8. The topical sunscreen composition of claim 5 wherein the topical sunscreen composition is a broad spectrum sunscreen.

9. The topical sunscreen composition of claim 5 wherein the topical sunscreen composition is transparent when applied.

10. A method of protecting a body from sun exposure comprising:
applying a topical sunscreen composition to a body and exposing the body to sun, the lotion including titanium dioxide, zinc oxide, water, avocado oil, sunflower oil, bee's wax, coconut oil, monolaurin, stearyl dimethicone, cetyl esters, ethyl linoleate, triethyl citrate, fragrance and Lecithin.

11. The method of claim 10 wherein the titanium dioxide is present in an amount of from 4 to 8% by weight, the zinc oxide is present in an amount of from 3 to 9% by weight, the water is present in an amount of from 21 to 44% by weight, the avocado oil is present in an amount of from 19 to 37% by weight, the sun flower oil is present in an amount of from 4% to 10% by weight, the bee's wax is present in an amount of from 3% to 6% by weight, the coconut oil is present in an amount of from 2 to 4% by weight, the monolaurin is present in an amount of from 2 to 4% by weight, the steryl dimethicone is present in an amount of from 2 to 4% by weight, the cetyl esters is present in an amount of from 1 to 3% by weight, the ethyl linoleate is present in an amount of from 1 to 3% by weight, the triethyl citrate is present in an amount of 1.5 to 2.5% by weight, the fragrance is present in an amount of 1 to 3% by weight, the Lecithin is present at about 0.5% by weight wherein the weight is based upon a total weight of the composition.

12. The method of claim 10 wherein the topical sunscreen composition has an SPF of 30 or greater.

13. The method of claim 10 wherein the topical sunscreen composition is a broad spectrum sunscreen.

14. A method of protecting a body from sun exposure comprising:
applying a topical sunscreen composition to a body and exposing the body to sun, the lotion including zinc oxide, water, avocado oil, zeolite, bee's wax, coconut oil, monolaurin, ethyl linoleate, bentonite, triethyl citrate, fragrance and Lecithin.

15. The method of claim 15 wherein the zinc oxide is present in an amount of from 3.85 to 10.15% by weight, the water is present in an amount of from 20 to 41% by weight, the avocado oil is present from 19 to 39% by weight, the zeolite is present in an amount of from 7% to 17% by weight, the bee's wax is present in an amount of from 2% to 4% by weight, the coconut oil is present in an amount of from 8 to 16% by weight, the monolaurin is present in an amount of from 8 to 16% by weight, the bentonite is present in an amount of from 4% to 8% by weight, the ethyl linoleate is present in an amount of from 1 to 3% by weight, the triethyl citrate is present in an amount of 1.5 to 2.5% by weight, the fragrance is present in an amount of 0.1 to 0.5% by weight, the Lecithin is present at about 0.2 to 0.4% by weight wherein the weight is based upon a total weight of the composition.

16. The method of claim 15 wherein the topical sunscreen composition has an SPF of 30 or greater.

17. The method of claim 15 wherein the topical sunscreen composition is a broad spectrum sunscreen.

18. The method of claim 15 wherein the topical sunscreen composition is transparent when applied.
